(19) United States
(12) Patent Application Publication
(10) Pub. No.: US 2021/0123087 A1
(43) Pub. Date: Apr. 29, 2021

(54) METHOD FOR TESTING ANTIMICROBIAL ACTIVITY OF A MATERIAL

(71) Applicant: University of South Alabama, Mobile, AL (US)

(72) Inventor: Terrence J. Ravine, Mobile, AL (US)

(21) Appl. No.: 17/076,130

(22) Filed: Oct. 21, 2020

Related U.S. Application Data
(60) Provisional application No. 62/973,784, filed on Oct. 25, 2019.

(51) Int. Cl. C12Q 1/18

(52) U.S. Cl. CPC C12Q 1/18

(57) ABSTRACT

A method for testing a sample for antimicrobial activity is disclosed. The method includes: (a) inoculating the sample with a pigment-competent microorganism; (b) incubating the sample at an incubation temperature for an incubation time period sufficient for growth of the pigment-competent microorganism and for pigment production; and (c) detecting the presence or absence of the pigment-competent microorganism on or in the sample. The method employs an easy-to-interpret color development approach to detect antimicrobial activity of sample materials such as biocidal fabrics.

### PRIORITY APPLICATION INFORMATION

The present application claims priority to U.S. Provisional Application Ser. No. 62/973,784 filed Oct. 25, 2019 and entitled “A NOVEL COLOR DEVELOPMENT ASSAY FOR SCREENING ANTIBACTERIAL ACTIVITY OF FABRIC TREATED WITH BIOCIDE AGENTS”; the entire contents and description of which is incorporated herein by reference.

### FIELD OF THE INVENTION

The present invention relates to an assay or method for testing the presence, absence or level of antimicrobial activity of materials or samples. The method uses an easy to interpret color development approach which may be referred to herein as “chromogenic”.

### BACKGROUND OF THE INVENTION

Materials with antimicrobial efficacy are known and useful in many industrial and consumer applications. For example, fabrics or textile materials that are coated, treated or functionalized to provide antimicrobial or antibacterial properties are an important topic in the textile industry. Generally, fabrics provide both a good contact area and can absorb moisture, each being a condition suitable for microbial growth. This growth can lead to fabric damage, spoilage and deterioration, malodors and the like and in apparel applications can cause dermal infections and even allergic responses. Thus, incorporation of antimicrobial agents on or in textile products to impart the ability to overcome these problems is of utmost importance.

Several standard test methods exist to assess antimicrobial activity of a textile product. Such methods include qualitative methods such as American Association of Textile Chemists and Colorists (AATCC) Test Method 147 titled “Antibacterial Activity Assessment of Textile Materials: Parallel Streak Method”. Other existing methods are quantitative methods as exemplified by ASTM E3160-18 Standard Test Method for Quantitative Evaluation of the Antibacterial Properties of Porous Antibacterial Treated Articles and AATCC 100 titled “Antibacterial Finishes on Textile Materials: Assessment of”.

Current textile industry standard methods that are qualitative in nature, e.g. AATCC 147, implement a nutrient agar (NA) plate diffusion method. These methods include placing a textile sample in direct contact with NA plates containing test bacterial cells. In the AATCC 147 method, samples are placed over the NA layer, previously streaked, i.e. five parallel streak protocol, with an inoculum of test bacterium. Similarly, in the ISO 20645 method, fabrics are positioned between two-layer NA plates—the lower layer only with NA and the upper layer inoculated with selected bacteria. Comparatively, in the JIS L 1902 method samples are placed on only one NA layer containing test bacterial cells. Such standard methods suggest the use of Gram-positive and Gram-negative bacteria species, Staphylococcus aureus or Klebsiella pneumoniae respectively, although other bacterial species can be used depending on the intended end-use of the tested fabric sample.

These qualitative methods evaluate the bacterial activity by the formation of a zone of inhibition (ZOI) immediately adjacent to fabric sample edges where the parallel streaks were placed and an examination underneath the sample for presence or absence of bacterial growth. A ZOI is created as diffusion of biocidal agent occurs away from the fabric sample into the surrounding NA and is measured to provide some indication of the potency of the antimicrobial activity of biocide treated textile samples, but cannot consistently be used as a quantification method. For example, a ZOI may not be seen with biocidal agents that remain tightly bound to fabric surfaces (since they do not readily diffuse into NA) though they still may be efficacious. After suitable incubation, the ZOI beyond the sample's edge along parallel streak lines is measured on both sides. Similarly, the contact area directly between the NA and the fabric sample is examined for bacterial growth, or its absence, along parallel streak lines made with the test bacteria. Standards generally state that in order to constitute acceptable antibacterial activity, there must be no bacterial colonies directly between the NA and the fabric sample in the parallel streak contact area. Bacterial growth, or its absence, between the NA and fabric sample may or may not be well defined (e.g., spotty growth). Likewise, the ZOI on either side of the fabric sample may not be equal, or isolated bacterial colonies may be seen growing up to the fabric edge even when a measurable ZOI is present. Additionally, the use of non-pigmented bacterium such as S. aureus and K. pneumoniae on NA can be problematic in detecting their growth on the front of or reverse of white or lightly colored fabric samples. These issues limit current standard test methods' utility in supplying consistent and conclusive antimicrobial test results.

Accordingly, and in response to the disadvantages experienced by the prior art, Applicant here provides for an antimicrobial test method for fabrics that is simple, effective and sufficiently sensitive to qualitatively detect bacterial growth. The present invention, unlike AATCC 147 for example, does not implement bacterial streaking on NA for diffusion of antimicrobial agent and ZOI determination method. Therefore, neither a ZOI measurement nor an examination for spotty (uneven) growth between a fabric sample and NA surface is required, and interpretation of results involves simple detection or visual inspection for the presence, absence or level of a color.

### SUMMARY OF THE INVENTION

In one aspect, the present invention is an assay or method for testing a sample for antimicrobial activity. The method of the present invention includes: (a) inoculating the sample with a pigment-competent microorganism; (b) incubating the sample at an incubation temperature for an incubation time period sufficient for growth of said pigment-competent microorganism and for pigment production; and (c) detecting the presence or absence of the pigment-competent microorganism on or in the sample.

In one or more embodiments of the method of the present invention, the sample includes a textile material or fabric and may be a biocidal material or a biocidal fabric or a biocidal textile material.

In one or more embodiments of the method of the present invention, the pigment competence of the pigment-competent microorganism is endogenous to the pigment-competent microorganism.

In one or more embodiments of the method of the present invention, the pigment-competent microorganism is a prodigiosin-competent microorganism is selected from the group consisting of Serratia marcescens, Pseudomonas magneslorubra, Vibrio psychroerythrous, Serratia rubidaea, Vibrio gazogenes, Alteromonas rubra, Rugamonas rubra and Gram-positive Actinomycetes, such as Streptoverticiffium rubrireticuli and Streptomyces longisporus ruber.

In one or more embodiments of the method of the present invention, the pigment-competent microorganism is a violacein-competent microorganism.

In one or more embodiments of the method of the present invention, the pigment-competent microorganism includes Chromobacterium violaceum.

In one or more embodiments of the method of the present invention, the prodigiosin-competent microorganism includes Serratia marcescens.

In one or more embodiments of the method of the present invention, the incubation temperature is from 20° C. and 45° C. or from 20° C. to 37° C.

In one or more embodiments of the method of the present invention, the incubation time period is between 6 and 30 hours.

In one or more embodiments of the method of the present invention, the step (c) of detecting the presence or absence of the pigment-competent microorganism on or in the sample includes visually inspecting the sample for the presence or absence of color resulting from the pigment-competent microorganism being present or absent on or in the sample.

In one or more embodiments of the method of the present invention, the method further includes as step (d) interpreting the results of detecting step (c).

In one or more embodiments of the method of present invention, the interpreting step (d) includes qualitatively assessing the antimicrobial activity of the sample or quantitatively measuring the antimicrobial activity of the sample.

Further aspects and embodiments of the invention are as disclosed and claimed herein. It should be understood that the description and specific examples are intended for purposes of illustration only and are not intended to limit the spirit and scope of the present invention.

### BRIEF DESCRIPTION OF THE DRAWINGS

The invention will be described in further detail below, and with reference to the accompanying drawings, wherein

FIG. 1 is a diagrammatic depiction of a general fabric sample arrangement in a polystyrene tissue culture 12-well microplate that might be useful in the practice of the method of the present invention wherein PC indicates a positive control; NC indicates a negative control; C1 indicates a first test sample prepared under a first set of conditions generally described as condition 1, and C2 indicates a second test sample prepared under a second set of conditions generally described as condition 2;

FIG. 2 is a diagrammatic depiction of the fabric sample arrangement in a polystyrene tissue culture 12-well microplate used in Example 1 set forth below, wherein generation of color as evidence of the presence of prodigiosin from a prodigiosin-competent microorganism (e.g., Serratia marcescens) is compared to controls or standard microorganisms such as K. pneumoniae or S. aureus using untreated samples shown as blank fabrics (BF); with the microplate containing untreated fabric samples inoculated with either 1.4×10⁶ bacteria/mL of K. pneumoniae (A2, A3), 1.4×10⁶ bacteria/mL of S. aureus (B2, B3), 2.0×10⁶ bacteria/mL of S. marcescens (C2, C3) and subsequently incubated at either 30° C. (for FIG. 5) or 37° C. (for FIG. 6);

FIG. 3 is a diagrammatic depiction of one fabric sample arrangement in a polystyrene tissue culture 12-well microplate used in Example 2 set forth below, inoculated using Muller-Hinton broth as the nutrient medium for the inoculum, wherein generation of color evidences of the presence of prodigiosin from prodigiosin-competent microorganisms Serratia marcescens (with sample location indicated by “Sm” in the Figure) and Chromobacterium violaceum (with sample location indicated by “Cv” in the Figure);

FIG. 4 is a diagrammatic depiction of a second fabric sample arrangement in a polystyrene tissue culture 12-well microplate used in Example 2 set forth below, inoculated using Trypticase Soy Broth (TSB) as the nutrient medium for the inoculum wherein generation of color evidences the presence of prodigiosin from prodigiosin-competent microorganisms Serratia marcescens (with sample location indicated by “Sm” in the Figure) and Chromobacterium violaceum (with sample location indicated by “Cv” in the Figure);

FIG. 5 is a diagrammatic depiction of the results from the test described in Example 1 below, performed in accordance with the method of the present invention and using the fabric sample arrangement shown in FIG. 2 with an incubation temperature of 30° C. and an incubation time period of approximately 24 hours;

FIG. 6 is a diagrammatic depiction of the results from the test described in Example 1 below, performed in accordance with the method of the present invention and using the fabric sample arrangement shown in FIG. 2 with an incubation temperature of 37° C. and an incubation time period of approximately 24 hours;

FIG. 7 is a diagrammatic depiction of the results from the test described in Example 2 below, performed in accordance with the method of the present invention and using the fabric sample arrangement shown in FIG. 3 with an incubation temperature of 30° C. and an incubation time period of approximately 24 hours;

FIG. 8 is a diagrammatic depiction of the results from the test described in Example 2 below, performed in accordance with the method of the present invention and using the fabric sample arrangement shown in FIG. 3 with an incubation temperature of 37° C. and an incubation time period of approximately 24 hours;

FIG. 9 is a diagrammatic depiction of the results from the test described in Example 2 below, performed in accordance with the method of the present invention and using the fabric sample arrangement shown in FIG. 4 with an incubation temperature of 30° C. and an incubation time period of approximately 24 hours;

FIG. 10 is a diagrammatic depiction of the results from the test described in Example 2 below, performed in accordance with the method of the present invention and using the fabric sample arrangement shown in FIG. 4 with an incubation temperature of 37° C. and an incubation time period of approximately 24 hours; and

FIG. 11 is a diagrammatic depiction of the results from the blinded (meaning that the person performing the test in unaware of the sample details so as to avoid prejudicial results) assay test described in Example 1 below and comparing antimicrobial activity of various biocidal samples (1.3.0×10⁶ bacteria/mL).

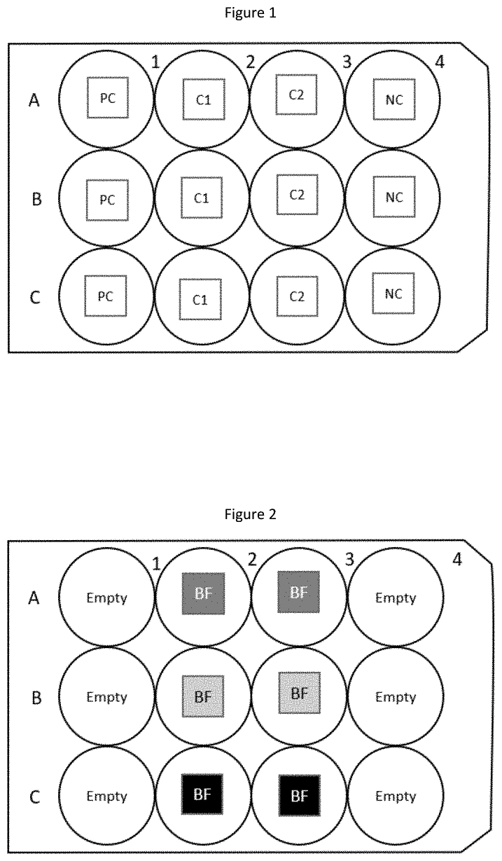

### DETAILED DESCRIPTION

The present invention represents a useful, improved and convenient method to test the level of antimicrobial activity of certain material samples. The method of the present invention includes: (a) inoculating the sample with a pigment-competent microorganism; (b) incubating the sample at an incubation temperature for an incubation time period sufficient for growth of said pigment-competent microorganism and for pigment production; and (c) detecting the presence or absence of the pigment-competent microorganism on or in the sample.

In one or embodiments, samples that may be tested according to the method of the present invention may be wettable samples, meaning that the sample is capable, e.g. through hydrophilic characteristics, surface tension and the like, to retain moisture sufficient to support microbial growth. Non-limiting examples of suitable samples include fabrics or textile materials. While the sample is wettable in one or more embodiments, one of ordinary skill will appreciate that materials and samples which are relatively or substantially hydrophobic or non-wettable may also be tested according to the method of the present invention by modifying their surface to retain moisture such as by placing a glass coverslip over the sample after the inoculating step described below. Sample size and shape may be selected based on a number of factors such as for example, number of samples tested, laboratory equipment configurations, use and selection of a particular test sample support or container and the like.

In one or more embodiments, the sample may include a biocidal material or a biocidal fabric or a biocidal textile material. The term “biocidal” is intended to include materials and samples which exhibit capacity to eliminate, inhibit the growth of or kill microorganisms such as for example viruses, bacteria, mycobacterium, fungus and the like. Biocidal may include without limitation antimicrobial, antifungal, antibacterial, antiviral and the like. Accordingly, antimicrobial activity tested by the method of the present invention may include for example biocidal activity, antibacterial activity, antifungal activity, antiviral activity and the like. Biocidal samples may include without limitation samples which are inherently biocidal because of their materials of construction; samples which are coated or treated and exhibit biocidal activity from the coating or treatment; and samples which are chemically functionalized and exhibit biocidal activity from the functionalization. In one or more embodiments, the sample may be a treated fabric or a coated fabric or a functionalized fabric. Samples and materials for their constructions are well known in the art and are described for example in WO 2020/086938A1, assigned to the assignee of the present invention; U.S. Published Patent Application No. 2015/0233049A1 and U.S. Pat. No. 7,291,570, the contents and disclosure of each of which are expressly incorporated herein by reference.

The method of the present invention includes the step of inoculating the sample with a pigment-competent microorganism or an inoculum that includes a pigment-competent microorganism. In the inoculating step, the sample is introduced to or otherwise exposed to or treated with the pigment-competent microorganism, typically in the form of an inoculum.

In one or more embodiments, the pigment-competent microorganism as a prodigiosin-competent microorganism. Prodigiosin is a known, red-pigmented bioactive secondary metabolite produced by certain Gram-negative and Gram-positive bacteria. “Prodigiosin-competent” is intended to include microorganisms which produce, express or similarly generate prodigiosin under certain conditions such as incubation. In one or more embodiments, the prodigiosin-competent microorganism is selected from the group consisting of Serratia marcescens, Pseudomonas magneslorubra, Vibrio psychroerythrous, Serratia rubidaea, Vibrio gazogenes, Alteromonas rubra, Rugamonas rubra and Gram-positive Actinomycetes, such as Streptoverticillium rubrireticuli and Streptomyces longisporus ruber. In a preferred embodiment, the prodigiosin-competent microorganism includes or consists essentially of or consists of Serratia marcescens.

In one or more embodiments, the pigment-competent microorganism is a violacein-competent microorganism. Violacein is a known, violet-colored bioactive secondary metabolite or pigment produced by certain Gram-negative and Gram-positive bacteria. “Violacein-competent” is intended to include microorganisms which produce, express or similarly generate violacein under certain conditions such as incubation. In one or more embodiments, the violacein-competent microorganism is selected from the group consisting of Chromobacterium violaceum, Janthinobacterium lividum, Collimonas spp., Duganella spp., Microbulbifer spp. and Pseudoalteromonas luteoviolacea. In a preferred embodiment, the violacein-competent microorganism includes or consists essentially of or consists of Chromobacterium violaceum.

It will be appreciated by one of ordinary skill that more than one pigment-competent microorganism may be utilized in inoculating step (a) and therefore that inoculating step (a) may include inoculating the sample with two or more pigment-competent microorganisms. By way of non-limiting example, the inoculating step (a) may include inoculating the sample with a prodigiosin-competent microorganism and a violacein-competent microorganism.

The method of the present invention may include the step of preparing the inoculum. The inoculum may be prepared by methods known to one of ordinary skill. In one suitable exemplary and non-limiting method for preparing an inoculum, a Mueller-Hinton (M-H) NA plate is inoculated with a single colony of S. marcescens and streaked for isolation (3-zone) and incubated for 24 hours at 30° C. under ambient conditions (no CO₂), Using an inoculating loop, sufficient well-isolated colonies are removed from the 24-hour plate and transferred to a tube containing 3 mL of Muller-Hinton broth (M-H) to approximate a 0.5% McFarland turbidity standard. Then, mix well and compare to standard. This will yield approximately 1×10⁸ CFU/mL⁻¹. Next, make a 1:100 dilution by adding 0.01 mL (10 μL) of 0.5% adjusted suspension to 0.99 mL (990 μL) of M-H broth and label tube as 10°. This should yield ˜1×10⁶ CFU/mL⁻¹. In another non-limiting example, a suspension of a pigmented-competent microorganism may be prepared in trypticase soy broth (TSB) to approximate a 0.5% McFarland turbidity standard to yield approximately 1×10⁸ bacteria/milliliter (mL) then a 1:100 dilution is made to reduce bacterial numbers to approximately 1.0×1 0\hat{ }6 bacteria/mL⁻¹.

One or ordinary skill will appreciate that the components and component amounts of the inoculum may vary and may be selected based on a number of factors such as for example identity and strain of target microorganism and incubation step conditions. In one or more embodiments, the inoculum includes a nutrient medium selected from the group consisting of Mueller-Hinton (M-H) broth/agar and trypticase soy broth/agar (TSB). In one or more embodiments, the inoculum includes trypticase soy broth/agar (TSB).

In one or more embodiments, the pigment competence of the pigment-competent microorganism may be endogenous to said pigment-competent microorganism. The term “endogenous” as used herein is intended to relate to a substance such as a pigment (and related competency) that is sourced from within the body of the microorganism. In one or more embodiments, the competence may be for example naturally endogenous in the sense that the microorganism sources the substance in its natural state or condition under conditions such as incubation. In one or more embodiments, the competence may be genetically induced in the sense that the microorganism is genetically modified or manipulated to source the substance under conditions such as incubation.

In one or more embodiments, the method of the present invention may include, prior to the inoculating step, a step of placing said sample into a test support or test container. A test support or test container may be any device or structure suitable for containing or supporting the sample or multiple samples during the subsequent method steps described herein. Non-limiting examples include culture plates such as multi-well culture plates and the like that may have 6, 12, 40 or any number of wells.

The method of the present invention further includes a step of incubating the sample at incubation conditions. Incubation conditions are generally conditions (including time, temperature, atmosphere, pressure etc.) which generate growth of the pigment-competent microorganism and pigment production by the pigment-competent microorganism. In one or more embodiments, the step includes incubation the sample at an incubation temperature for an incubation time period sufficient for growth of the pigment-competent microorganism and for pigment production. “Incubating” therefore describes subjecting the sample to conditions (including time, temperature, atmosphere, pressure etc.) which generate both growth of the pigment-competent microorganism and pigment production by the pigment-competent microorganism. Incubation time period and incubation temperature may vary based on a number of factors, including selection and strain of pigment-competent microorganism, inoculum composition, inoculum nutrient broth and the like. In one or more embodiments, the incubation temperature may range from 20° C. to 45° C. or from 20° C. to 37° C. or from 20° C. to 35° C. or from 20° C. to 32° C. In one or more embodiments, the incubation time period may range from 6 hours to 30 hours. In a preferred embodiment, the incubating step may occur at an incubation temperature of 30 degrees Centigrade under ambient surrounding air conditions for an incubation time period of from 18 to 24 hours. Once the incubation time period has ended, the incubation step can be terminated.

The method of the present invention further includes a step of detecting the presence or absence of the pigment-competent microorganism on or in the sample. In one or embodiments, the detecting step may include visually inspecting the sample for the presence or absence of color resulting from the pigment-competent microorganism being present or absent on or in said sample. In one more embodiments, the detecting step may include optically inspecting the sample, for example with an automated optical device detecting light wavelength reflectance (such as a reflectometer) or absorption, for the presence or absence of color resulting from resulting from the pigment-competent microorganism being present or absent on or in the sample. The detecting step may include detecting wavelengths of light energy reflected from the sample. In one or more embodiments, the detecting step may include detecting wavelengths of light energy of between 570 nm and 750 nm reflected from the sample. More generally, the detecting step may alternatively be described as a step of detecting the presence or absence of color resulting from the pigment-competent microorganism being present or absent on or in the sample. In one or more embodiments, the detecting step may include detecting wavelengths of light energy of between 380 nm and 500 nm reflected from the sample.

Though in many embodiments the detecting step may include detecting the presence or absence of color, it will be appreciated by one of ordinary skill that the presence or absence of color on or in the sample may manifest itself in various shades of gray, for example to a colorblind person visually inspecting the sample or to an optical device measuring lightness or darkness rather than color. It is to be understood that “color” as the term is used herein may also encompass shades of lightness and darkness between black and white.

In one or more embodiments, the method of the present invention may further include a step (d) of interpreting the results of said detecting step. Generally, the step of interpreting the results may include comparing the color level of the sample to a control sample and/or a sample of known antimicrobial activity. The step of interpreting the results may generally include labeling the sample antimicrobial activity as present or absent or acceptable or unacceptable for a given application or based on a set of criteria. In one or more embodiments, interpreting step (d) includes qualitatively assessing the antimicrobial activity of said sample. In one or more embodiments, interpreting step (d) includes quantitatively measuring the antimicrobial activity of said sample. In one or more of these embodiments, the quantitively measuring step includes measuring emitted or reflected wavelengths of light energy from said sample. In one or more of these embodiments, the quantitively measuring step includes measuring wavelengths of light energy of between 570 nm and 750 nm or of between 380 nm and 500 nm reflected from the sample.

The following examples, while provided to illustrate with specificity and detail the many aspects and advantages of the present invention, are not to be interpreted as in any way limiting its scope. Variations, modifications and adaptations which do depart of the spirit of the present invention will be readily appreciated by one of ordinary skill in the art.

### Example 1

### Introduction

The present invention uses an easy to interpret color development approach to detect antimicrobial activity of sample materials such as biocidal fabrics. In one or more embodiments, the method of the present invention utilizes the prodigiosin competency of a prodigiosin-competent microorganism such as the bacterium S. marcescens. In one or more embodiments, the method of the present invention uses the violacein competency of a violacein-competent microorganism such as Chromobacterium violaceum. Neither K. pneumoniae nor S. aureus can (ATTCC) produce similar color (pigment) when incubated at an incubation temperature such as 30° C. or 37° C. It should also be noted that some strains of S. marcescens may produce a slight amount of light pink color when incubated at 37° C.

The presence of color may in general be characterized by the detection, visible or otherwise, of any point on the color spectrum of a color. Examples include, for the primary color red, any intensity of red shade, e.g. “pink”, “crimson” and “rose”; and for the color violet, any intensity of violet shade such as e.g. “purple”, “lavender”, “magenta”, “indigo”, “dark blue” and the like. The absence of color is characterized by the inability to detect visibly or otherwise any point on the color spectrum. By way of an example that can be helpful in the practice of the method of the present invention, an untreated or blank fabric sample that is not intrinsically antimicrobial or treated or functionalized with a biocidal material may be white in color, but when treated with an inoculum of S. marcescens and incubated so as to grow and produce the red-pigment prodigiosin, may exhibit a red color indicative of the sample being positive (+) for bacterial growth as shown in the Figures. A sample generally exhibiting a white color after incubation may be indicative of the absence of pigment and the pigment-competent microorganism and are therefore shown as negative (−) for bacterial growth as shown the Figures. As such, the presence of a color after incubation may be interpreted as the sample being positive (+) for bacterial growth and the material represented by sample may be deemed deficient in its antimicrobial properties or antibacterial activity. Conversely, the presence of a white color after incubation, or more precisely the absence of a color, may be interpreted as the sample being negative (−) for bacterial growth and the material represented by sample may be deemed sufficient in its antimicrobial properties or antibacterial activity. If a color of lighter shade or depth detected, antimicrobial activity may be present but not sufficient to completely retard or eliminate bacterial growth and is shown by a (±) in Figures. Result interpretation may be a binary function where either pigment-competent microorganism growth or no growth is seen as visualized by pigment production.

Test Methods

In a first test example of the method of the present invention, labeled Example 1, an inoculum was prepared that included S. marcescens bacterial suspension in Muller-Hinton broth (M-H) matching a 0.5% McFarland turbidity standard. This yielded approximately 1×10⁸ bacteria/milliliter (mL). Next, a 1:100 dilution of the suspension was made to reduce bacterial numbers and yielded approximately 1.0×10⁶ bacteria/mL. 12 mm×12 mm fabric sample squares were then cut and distributed into separate wells of a 12-well microplate. To inoculate the samples, and a 50 microliter (A) drop of inoculum in the form of the 1:100 dilution of bacterial suspension was then added to the center of each fabric sample.

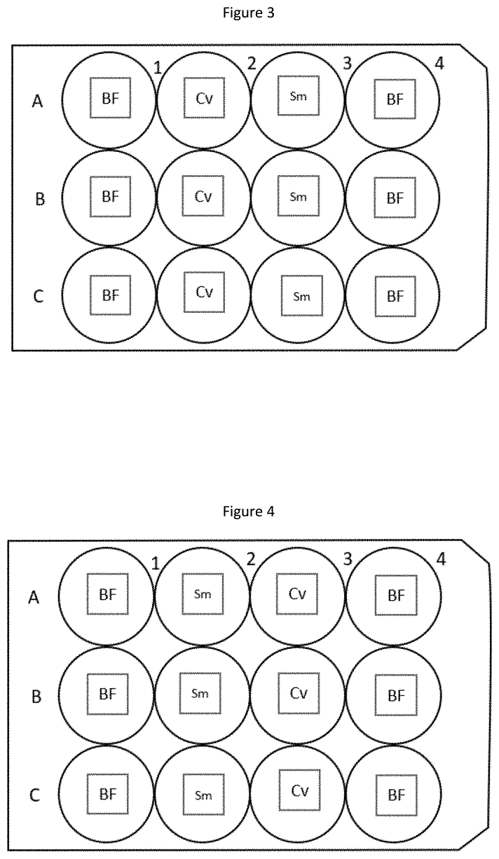

For Example 1, and as depicted in FIGS. 2, 5 and 6, a testing protocol was utilized to demonstrate in particular the utility of the prodigiosin-competent microorganism when compared to microorganism utilized in other AATCC test protocols. In this aspect of the example, inoculums of K. pneumoniae and S. aureus were prepared in a manner similar to that described above for S. marcescens and the samples inoculated with the inoculums as follows: wells A2 and A3 with K. pneumoniae inoculum; B2 and B3 with S. aureus inoculum and C2 and C3 with S. marcescens inoculum. The cells in numbered columns 1 and 4 were left empty as indicated in FIG. 2. Two microplates with duplicate sample arrangements as shown in FIG. 2 were prepared, with the first microplate then incubated at 30° C. under ambient surrounding air conditions for approximately 24 hours and the second microplate incubated at 37° C. under ambient surrounding air conditions for approximately 24 hours. After incubation was complete, the samples were visually inspected for red color with results logged as either positive (+) for red color indicating growth or negative (−) for red color indicating no growth as shown in FIG. 5 for the microplate incubated at 30° C. and FIG. 6 for the microplate inoculated at 37° C. As shown in FIG. 5, the method of the present invention can achieve an easily visible indication of bacterial detection. The results depicted in FIG. 6 indicated that the specific strain of S. marcescens tested did not produce a significant amount of prodigiosin at the tested temperature of 37° C. From this result, one of ordinary skill will appreciate that process variables such as for example incubation conditions, microorganism selection, specific microorganism strain, inoculum composition (including nutrient medium) should be selected and aligned to ensure and preferably optimize the potential for both microorganism growth and pigment production under the selected incubation conditions.

In a separate aspect of this Example 1, all twelve cells of a 12-cell microplate were utilized to demonstrate the method of the present invention and to discern and evaluate the antimicrobial properties of various fabric samples. A 12 mm×12 mm sample was inserted into each well, inoculated with an S. marcescens inoculum prepared as described above and incubated at 30° C. for 24 hours under ambient surrounding air conditions. After incubation was complete, the samples were visually inspected for red color with results logged as either positive (+) for red color indicating bacterial growth, negative (−) for red color indicating the absence of bacterial growth or as shown in FIG. 11.

As shown in Example 1 and the related Figures, the method of the present invention provides numerous benefits and advantages. The method of the present invention eliminates subjective interpretation of ZOI and/or growth underneath samples by detecting bacterial growth via a convenient visual inspection and detection of the presence or absence of color through use of a prodigiosin-competent microorganism. Further, the method of the present invention creates pigmented or stained samples that can provide a semi-permanent record of results. Also, the method of the present invention provides for simultaneous testing and side-by-side comparative assessment of multiple samples. Further, the method of the present invention utilizes a reduced sample footprint or area (less than 1250 mm² or less than 1000 mm² or less than 500 mm² or no more than 150 mm²) versus prior art protocols and creation of pigment (color) permanently stains fabric samples providing a semi-permanent record of results.

### Example 2

In Example 2, two separate tests were conducted using inoculums of different pigment-competent microorganisms prepared using the general procedure set forth in Example 1. These tests were performed in part to determine if color development might be impacted by substituting trypticase soy broth for Mueller-Hinton broth as the nutrient medium in the inoculum. Both tests were conducted under the same conditions. For all Example 2 testing, multiple 12 mm×12 mm samples of untreated cotton fabric (referred to a “blank” fabric or BF) were cut from a larger piece of material with one sample each placed in each cell of a 12-cell microplate, initially without inoculation.

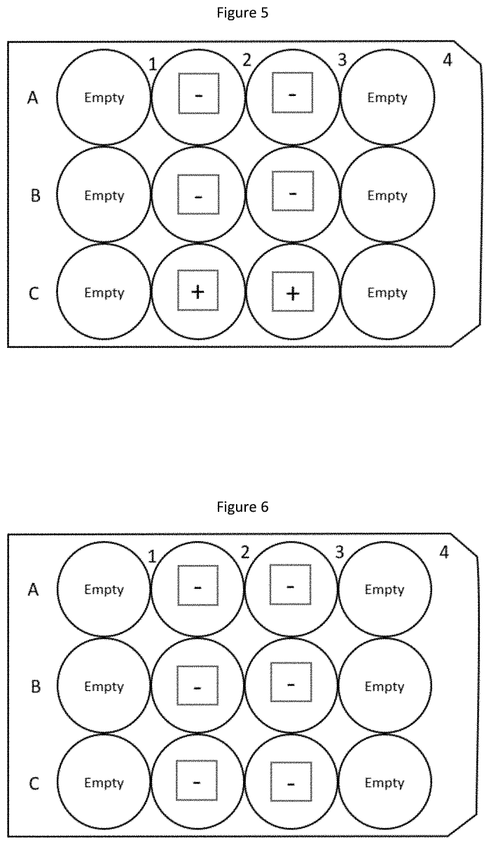

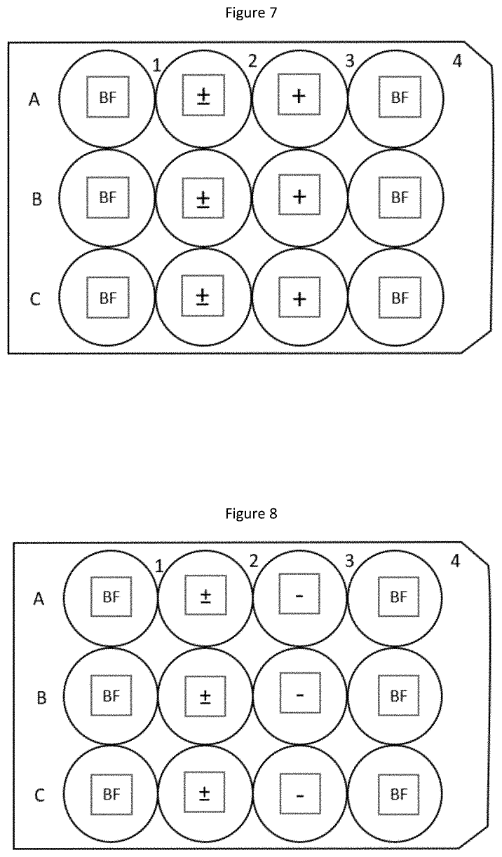

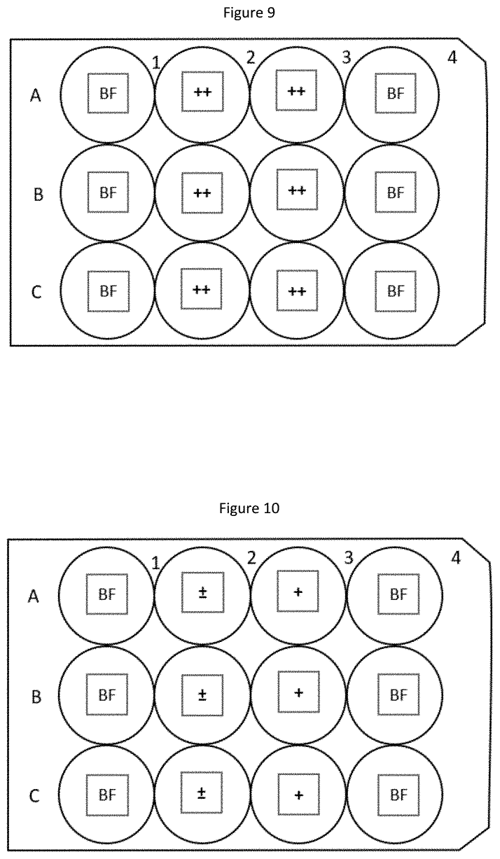

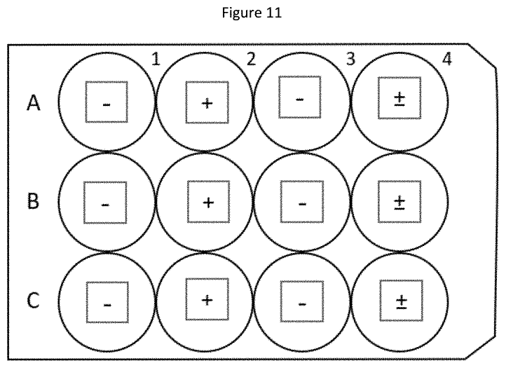

In test condition 1, samples A3-C3 of the sample fabric arrangements as shown in FIG. 3 were inoculated with a first inoculum that included S. marcescens (final yield 1.4×10⁶ CFU/mL) while samples A2-C2 of the sample fabric arrangements as shown in FIG. 3 were inoculated with a second inoculum that included C. violaceum (final yield 0.3×10⁶ CFU/mL), both suspended in Mueller-Hinton broth. In test condition 2, a first inoculum included S. marcescens (final yield 1.3×10⁶ CFU/mL) and a second inoculum included C. violaceum (final yield 0.7×10⁶ CFU/mL), both suspended in trypticase soy broth. As shown in FIG. 4, samples inoculated with the Chromobacterium violaceum-containing inoculum were placed in cells A3-C3 and samples inoculated with the S. marcescens-containing inoculum were placed in cells A2-C2. Two microplates with duplicate sample arrangements as shown in FIGS. 3 and 4 were prepared for each broth/inoculum, with the first microplate(s) then incubated at 30° C. under ambient surrounding air conditions for approximately 24 hours and the second microplate(s) incubated at 37° C. under ambient surrounding air conditions for approximately 24 hours. After incubation was complete, the samples were visually inspected for color with results logged as positive (+) for color indicating growth; negative (−) for color indicating no growth; or multiple positive or negative logs to indicate depth and degree of color as shown in FIGS. 7 and 9 for the microplates incubated at 30° C. and FIGS. 8 and 10 for the microplates incubated at 37° C. As shown in particular in FIGs, the method of the present invention may achieve an easily visible bacterial detection for both S. marcescens and Chromobacterium violaceum samples, with the depth and intensity of the color shades for the samples inoculated with the S. marcescens-containing inoculum and incubated at 37° C. lighter than that incubated at 30° C. It further demonstrates that Chromobacterium violaceum is a sustainable growth indicator at either 30° C. or 37° C. incubation temperatures. Finally, color and shade depth appears markedly improved by substituting TSB for M-H broth as nutrient medium for the inoculum, in particular at the 30° C. incubation temperature and for samples with the S. marcescens-containing inoculum.

Precision and Bias

Using a 0.5% McFarland turbidity standard to make the initial bacterial inoculum helps to eliminate variability in results between laboratories; however, the precision and bias for the test method as described in this Example was not determined.

One of ordinary skill will appreciate that performing bacterial plate counts, in conjunction with the method of the present invention, may help to reduce quantitative variability of results between runs due to differing amounts of viable bacteria being present in the initial inoculums. An example of a suitable plate count method is below.
1. Prepare four ten-fold serial dilutions (10⁻¹, 10⁻², 10⁻³, 10⁻⁴) using the 100 tube (1:100 dilution) of standardized inoculum in 0.9% saline. Distribute 0.9 mL sterile saline to four tubes labeled with the above dilutions.
2. Add 0.1 mL (100 μL) of the 100 tube contents to 0.9 mL (900 μL) of sterile saline in the 10-1 tube. Repeat process for the remaining tubes.
3. Transfer 0.1 ml of the 10⁻³ dilution to a fresh M-H NA plate and spread drop over NA surface using a sterile cell spreader. Repeat process for 10⁻⁴ dilution. Incubate both plates at 30° C. for 24 hours.
4. Remove 10⁻³ and 10⁻⁴ count plates and count the number of colonies on the 10⁻⁴ plate. Use the 10⁻³ plate for counting if less than 10 colonies are present on the 10⁻⁴ plate.
5. Calculate the number of colony forming units (CFU)/mL using the below formula.

Colonies counted×reciprocal of tube dilution×10 (dilution factor)=total CFU/mL⁻¹

### Example 3

Applicant submits that the below illustrative example of an embodiment of the method of the present invention in the prospective form of an AATCC protocol format may be helpful to the person of ordinary skill in the practice of the method of the present invention.

### Antimicrobial Activity Assessment of Textile Materials

### Serratia marcescens Chromogenic Plate Method

Forward

This Chromogenic Plate Method represents a relatively quick and conveniently executed qualitative method to determine antibacterial activity on biocidal materials, more particularly treated textile materials.

AATCC Test Method 147, Antibacterial Activity Assessment of Textile Materials: Parallel Streak Method, is a qualitative procedure which is dependent on antimicrobial agent diffusion from the sample into the surrounding agar, can generate variable results, requires subjective interpretation, and is technique-dependent for a routine qualitative antimicrobial screening test. Therefore, when the intent is to demonstrate antibacterial activity independent of the diffusion characteristics of the antibacterial agent, with less cumbersome research tools and reagents, allowing internal control samples in small- or large-scale sample testing, the present invention as exemplified in this Serratia marcescens Chromogenic Plate Method fills this need. The Serratia marcescens Chromogenic Plate Method has proven effective over a number of preliminary studies in providing evidence of antibacterial activity, or absence thereof, for biocidal fabric material test samples.

### 1. Purpose and Scope

1.1 The objective is to detect antimicrobial activity on biocidal materials. The method is useful for obtaining an estimate of activity in that the growth of the inoculum organism is determined by the presence of the signature red-like color of the red-pigment prodigiosin that is produced by viable prodigiosin-competent microorganism, e.g. S. marcescens. The absence of red color on or in the textile material test sample affected for example by the presence of an antibacterial agent permit an estimate of the level of antibacterial activity in or on the textile material.

### 2. Principle

This method takes advantage of the ability of the Gram-negative bacterium S. marcescens to produce a red pigment called prodigiosin. Specimens of the test materials, including corresponding untreated controls of the same material as desired, are placed in a container, e.g polystyrene multi-well plate, and the test materials are inoculated with a standardized amount of test bacterium. After incubation, the presence of any shade of red color, i.e. the presence of color, on the test material is interpreted as the sample being positive (+) for bacterial growth while a white color, i.e. the absence of red color, is interpreted as the sample being negative (−) for bacterial growth. If a red color of lighter shade or depth detected, antimicrobial activity may be present but not sufficient to completely retard or eliminate bacterial growth and may be labeled by a (±).

### 3. Terminology

1.1 activity, n.—of an antibacterial agent, a measure of effectiveness of the agent
1.2 antibacterial agent, n.—in textiles, any chemical which kills bacteria (bactericide) or interferes with the multiplication, growth or activity of bacteria (bacteriostat)
1.3 presence of color, n.—of prodigiosin in origin, visible appearance of any shade along the color spectrum of red

NOTE: The “presence of red color” on a textile test sample occurs as a result of the production of the red pigment prodigiosin by live and growing bacterium, indicating an ineffectiveness of the antimicrobial agent.

### 4. Safety Precautions

NOTE: These safety precautions are for information purposes only. The precautions are ancillary to the testing procedures and are not intended to be all inclusive. It is the user's responsibility to use safe and proper techniques in handling materials in this test method. Manufacturers MUST be consulted for specific details such as material safety data sheets and other manufacturer's recommendations. All OSHA standards and rules must also be consulted and followed.
4.1 This test should be performed only by trained personnel. The U.S. Department of Health and Human services publication Biosafety in Microbiological and Biomedical Laboratories should be consulted.
4.2 CAUTION: Some of the bacteria used in this test may be pathogenic; i.e., capable of infecting humans and producing disease. Therefore, every necessary and reasonable precaution must be taken to eliminate this risk to the laboratory personnel and to personnel in the associated environment. Wear protective clothing and respiratory protection that prevents penetration by the bacteria.
4.3 Good laboratory practices should be followed. Wear safety glasses in all laboratory areas.
4.4 All chemicals should be handled with care.
4.5 An eyewash/safety shower should be located nearby for emergency use.
4.6 Sterilize all contaminated samples and test materials prior to disposal.
4.7 Exposure to chemicals used in this procedure must be controlled at or below levels set by government authorities (e.g., Occupational Safety and Health Administrations [OSHA] permissible exposure limits [PEL] as found in 29 CFR 1910.1000 of Jan. 1, 1989). In addition, the American Conference of Governmental Industrial Hygienists (ACGIH) Threshold Limit Values (TLVs) comprised of time weighted averages (TLV-TWA), short term exposure limits (TLV-STEL) and ceiling limits (TLV-C) are recommended as a general guide for air contaminant exposure which should be met.
4.8 As a general matter, aseptic (sterile) techniques should be employed during the handling of bacterial organisms.
4.9 Suitable contamination control protocols should be included to ensure purity of tested bacterial inoculum.

### 5. Uses and Limitations

The method may not be suitable for materials which require testing for diffusion of the antibacterial agent.

### 6. Test Organisms

6.1 Test Bacteria:
   6.1.1 Serratia marcescens, a Gram-negative prodigiosin-competent microorganism or Chromobacterium violaceum, a violacein-pigment competent microorganism.
   6.1.2 Other pigment-competent species can also be used.
6.2 Whenever possible, test the activity of the culture to be used against a standard control specimen (a positive control) with known antibacterial activity.
6.3 To determine whether the antibacterial activity is due to the antibacterial agent, test a specimen of the same material treated in exactly the same way with whatever other finishing agents were used, but without the antibacterial agent. Many standard textile finishing chemicals, especially crease resistant and permanent press reagents, will often give strong antibacterial activity even after many washes.

### 7. Culture Medium

7.1 Suitable broth/agar media is Mueller-Hinton (M-H) or trypticase soy broth (TSB).
7.2 Heat to a boil to disperse ingredients. Adjust to specific pH with NaOH solution. (This is not necessary if prepared, dehydrated medium is used.)
7.3 Add 1.5% bacteriological agar to nutrient (or appropriate) broth. Heat to boiling. Check pH and adjust to using NaOH solution if necessary. Dispense in appropriate amounts in conventional bacteriological culture tubes, plug, and sterilize at 103 kPa (15 psi) for 15 min. (May be sterilized in 1,000 mL borosilicate glass flasks and petri dishes poured from this.)

### 8. Maintenance of Culture of Test Organisms

8.1 Inoculate using aseptic (sterile) technique a Mueller-Hinton (M-H) agar plate or trypticase soy agar (TSA), with or without addition of 5% sheep's blood, with a single colony of S. marcescens and streak for isolation (3-zone). Incubate for 24 hours at 30° C. under ambient conditions, no CO₂ required.
8.2 Using a 4 mm inoculating loop, transfer the culture daily in nutrient (or appropriate medium) broth for not more than two weeks. At the conclusion of two weeks, make a fresh transplant from stock culture. Incubate cultures at 32±2° C. (86±3° F.).
8.4 Maintain stock cultures on nutrient on agar plates or appropriate agar nutrient broth slants. Store at 4° C. to 8° C. and transfer once a month to fresh agar. Cultures can also be frozen at −20° C. or −70° C. when suspended in freezing medium for long term storage.

### 9. Test Specimens

Test specimens (non-sterile) are cut by hand or with a die. They may be any convenient size. Rectangular specimens cut 12×12 mm are recommended. A 12 mm length and width permits the specimen to lie flat within the well of a standard 12-well polystyrene plate. Smaller samples may be positioned in a 40-well plate to increase the number of sample replicates or test conditions. This is an acceptable practice as long as like visible color detection is supported

### 10. Procedure

10.1 Inoculate a Mueller-Hinton (M-H) or trypticase soy broth (TSB) agar plate with a single colony of S. marcescens or C. violaceum and streak for isolation (3-zone). Incubate for 24 hours at 30° C. under ambient conditions (no CO₂).
10.2 Prepare inoculum by transferring via inoculating loop enough well isolated bacterial colonies from the 24-hour plate and transfer to a tube containing 3 mL of Muller-Hinton broth (M-H) to approximate a 0.5% McFarland turbidity standard. Mix well and compare to standard. This will yield approximately 1×10⁸ CFU/mL⁻¹.
10.3 Make a 1:100 dilution by adding 0.01 mL (10 μL) of 0.5% adjusted suspension to 0.99 mL (990 μL) of M-H broth and label tube as 100. This should yield ˜1×10⁶ CFU/mL⁻¹.
10.4 Prepare samples by situating test and any control samples square flat against the bottom of each well in a 12-well culture plate. Multiple culture plates may be used to accommodate a larger sample number, if desired.
10.5 Add a 50 μL drop of 100 tube (1:100 dilution) of bacterial suspension to center each fabric sample (change tips in between each sample). Tap fabric lightly with sterile forceps or a sterile wooden applicator stick to ensure that all parts of wetted fabric make good contact with well bottom. A separate sterile device may be used for each condition or devices such as forceps may be cleaned with methanol and dried as needed between samples. Incubate culture plate(s) @ 30° C. under ambient air conditions (without CO₂) for 18-24 hours.

### 11. Evaluation

11.1 Visibly examine the incubated samples for the presence, visible or otherwise, of any shade intensity or depth on the color spectrum of the primary color red, e.g. “pink”, “rose”, “crimson”.
11.2 The presence of any shade of red color is interpreted as the sample being positive (+) for bacterial growth, hence the fabric sample is deficient in its antimicrobial properties and the antibacterial activity is insufficient to prevent bacterial growth, while the absence of any shade of red color, is interpreted as the sample being negative (−) for bacterial growth, hence the fabric does exhibit antimicrobial properties and the antibacterial activity is sufficient to prevent bacterial growth.
11.3 The shade and saturation of the red color is not necessarily to be construed as a quantitative evaluation of antibacterial activity. Treated materials should be compared to an untreated corresponding material and a material specimen with known antimicrobial activity if available. Report of results will include an observation of presence or absence, shade and saturation of any color along the red color spectrum, if present. The criterion for passing the test (i.e. demonstrating acceptable antimicrobial activity) must be based on criteria and protocols agreed upon by the interested parties. To constitute acceptable antibacterial activity, there typically may be no [or minimal] presence of any shade of red in or on the sample.

### 12. Precision and Bias

Optionally, performing bacterial plate counts, in conjunction with the fabric assay, can help to reduce variability of results between runs due to differing amounts of viable bacteria being present in the initial inoculums.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method for testing a sample for antimicrobial activity, said method comprising:
a) inoculating said sample with an inoculum that includes a pigment-competent microorganism;
b) incubating said test sample at an incubation temperature for an incubation time period sufficient for growth of said pigment-competent microorganism and for pigment production; and,
c) detecting the presence or absence of said pigment-competent microorganism on or in said test sample.

2. The method of claim 1 further comprising, prior to said inoculating step, placing said sample into a test support.

3. The method of claim 1 wherein said sample comprises a biocidal material.

4. The method of claim 3 wherein said sample is a biocidal fabric or biocidal textile material.

5. The method of claim 1 wherein pigment competence for said pigment-competent microorganism is endogenous to said pigment-competent microorganism.

6. The method of claim 1 wherein said pigment-competent microorganism is a prodigiosin-competent microorganism.

7. The method of claim 6 wherein said prodigiosin-competent microorganism is selected from the group consisting of Serratia marcescens, Pseudomonas magneslorubra, Vibrio psychroerythrous, Serratia rubidaea, Vibrio gazogenes, Alteromonas rubra, Rugamonas rubra and Gram-positive Actinomycetes, such as Streptoverticillium rubrireticuli and Streptomyces longisporus ruber.

8. The method of claim 1 wherein said pigment-competent microorganism is a violacein-competent microorganism.

9. The method of claim 8 wherein said violacein-competent microorganism is selected from the group consisting of Chromobacterium violaceum, Janthinobacterium lividum, Collimonas spp., Duganella spp., Microbulbifer spp. and Pseudoalteromonas luteoviolacea.

10. The method of claim 9 wherein said violacein-competent microorganism comprises or consists essentially of or consists of Chromobacterium violaceum.

11. The method of claim 1 wherein said incubation temperature is from 20° C. to 37° C.

12. The method of claim 1 wherein said detecting step (c) comprises visually inspecting said sample for the presence or absence of color resulting from said prodigiosin-competent microorganism being present or absent on or in said sample.

13. The method of claim 1 wherein said detecting step (c) comprises optically inspecting said sample for the presence or absence of color resulting from said prodigiosin-competent microorganism being present or absent on or in said sample.

14. The method of claim 1 wherein said method further comprises, as step (d), interpreting the results of said detecting step.

15. The method of claim 1 wherein said interpreting step (d) comprises qualitatively assessing the antimicrobial activity of said sample.

16. The method of claim 1 wherein said interpreting step (d) comprises quantitatively measuring the antimicrobial activity of said sample.

17. The method of claim 13 wherein said quantitively measuring step comprises measuring wavelengths of light energy reflected from said sample.

18. The method of claim 13 wherein said quantitively measuring step comprises measuring wavelengths of light energy of between 380 nm and 500 nm reflected from said sample.
